(19) United States
(12) Patent Application Publication
(10) Pub. No.: US 2005/0175621 A9
(43) Pub. Date: Aug. 11, 2005

(54) Adjuvant composition comprising FHA protein or a fragment of FHA protein in its free form

(76) Inventors: Andre Capron, Phalempin (FR); Camille Locht, Bruxelles (BE); Franco Menozzi, Mons-Hyon (BE); Odile Poulain-Godefroy, Lambersart (FR); Gilles Riveau, Phalempin (FR)

Correspondence Address: NIXON & VANDERHYE, PC, 901 NORTH GLEBE ROAD, 11TH FLOOR, ARLINGTON, VA, 22203, US

(21) Appl. No.: 10/314,440

(22) Filed: Dec. 9, 2002

Related U.S. Application Data
(63) Continuation of application No. PCT/FR01/01769, filed on Jun. 7, 2001.

(30) Foreign Application Priority Data
Jun. 7, 2000 (FR) 0007302

(51) Int. Cl. A61K 39/00; A61K 39/38

(52) U.S. Cl. 424/184.1

(57) ABSTRACT

The invention concerns the use of the FHA protein or part of the FHA protein, in free form, as adjuvant of the immune response or as immunostimulant in a human or an animal. The invention also concerns adjuvant and immunostimulatory compositions comprising FHA protein or a fragment thereof, and the use of said compositions for making vaccines.

The present invention relates to the field of adjuvants for vaccination, and more particularly adjuvants that can stimulate immunity both mucosally and systemically.

The present invention concerns any use of the FHA protein or a portion of the FHA protein in its free form for the preparation of a composition adjuvanting the immune response for human or animal use.

It also relates to any composition adjuvanting the immune response comprising the free form of the FHA protein or a portion of the FHA protein.

It also concerns immunogenic and/or vaccine compositions comprising said adjuvant composition in association with an immunogen or an antigen.

Filamentous haemagglutinin (FHA) from Bordetella pertussis is a major adhesin produced and secreted by the bacterium. The structural gene for FHA codes for a 367 kDa protein, and the mature form is constituted by the 60 N-terminal % of the precursor (references 1 to 5).

The functions of the N-terminal and C-terminal portions of the precursor (fhaB) and that of the mature secreted protein (FHA) have been described by Geneviève Renauld-Mongénie et al., (6). It appears that the N-terminal domain of fhaB plays an essential role in the secretion of the mature protein since phase deletion in that region appears to completely inhibit that secretion.

In the mature state, the FHA protein has a molecular weight of 220 kDa and presents at least three characteristic binding sites (1):
a carbohydrate binding site which allows it to fix to ciliated epithelial cells;
a heparin and sulphated carbohydrate binding site involved in attachment to non ciliated epithelial cells and to the extra-cellular matrix;
a RGD sequence which allows attachment to macrophages via their integrins.

FHA is an adhesin produced by different stains of Bordetella. Particular examples that can be cited are the filamentous adhesins produced by Bordetella bronchiseptica and Bordetella parapertussis. The different FHA proteins of Bordetella are at least 60% homologous with the FHA sequence of Bordetella pertussis. Further, the sequences in the N-terminal region of FHA are very similar to the N-terminal domains of S. marcenscens or P. mirabilis (4).

Throughout the present text, any filamentous haemagglutinin with a sequence homology of at least 60% with the FHA of B. pertussis or any polypeptide with a sequence homology of at least 60% and preferably 70% with the FHA of B. pertussis must be considered to be a functional equivalent of the FHA of B. pertussis.

The adherence properties of the FHA to different cell types such as ciliated or non ciliated epithelial cells or monocytes/macrophages have been exploited in the prior art to target the presentation of an antigen of interest into cells which may be responsible for the presentation of the antigen to the immune system, possibly after maturation.

More particularly, the properties of the filamentous haemagglutinins of Bordetella to fix to non ciliated epithelial cells and to the extracellular matrix allows targeting to the mucosa to be envisaged.

Mucosal immunization offers certain advantages over parenteral immunization. Firstly, mucosal immunization simultaneously stimulates both mucosal immunity and systemic immunity, which is not the case with systemic immunization. Further, the mucosal route is an administration type that causes few secondary effects. Finally, the development of vaccines adapted to the demands of developing countries will definitely shift to mucosal administration. However, the majority of antigens are weak immunogens when administered mucosally, probably because their interaction with the mucosal immune system is weak. Different strategies have been developed to augment the immunogenicity of antigens administered by this route, firstly by encouraging their access to the mucosa, and secondly by using processes that can augment the response obtained.

Mucosal access is encouraged by antigens of a particular type in which interaction with the lymphoid tissues is facilitated. The most popular particular forms belong to a number of types: liposomes (7), microspheres (8), ISCOMs (9). They are all synthetic globular structures which encapsulate the antigen to be administered, with compositions and modes of preparation that differ according to their nature (lipids for liposomes, organic polymers for the microspheres, a mixture of lipids and Quil A for ISCOMs). A further approach consists of using recombinant microorganisms expressing the antigen of interest. Those microorganisms can be live or deactivated, and are used as a vector that supplies the antigen to the immune system behind the mucosa. Salmonella has been used as a live vector to initiate oral immunization. A live vector adapted to immunization via the respiratory tract has been produced by dint of the development of recombinant strains of Bordetella pertussis. Such strategies using live vaccines also have the advantage of allowing prolonged exposure of the antigen to the mucosa of the host during colonization and thus do not require the use of adjuvants.

Effective mucosal adjuvants have been developed in the first instance by testing adjuvants that have been proved to be effective parenterally, such as compounds from the muramyl-pepide family. Currently, the most effective mucosal adjuvants remain bacterial toxins such as cholera toxin, the E. coli toxin, and pertussis toxin to a lesser extent. One of the major problems in using such toxins as mucosal adjuvants is their toxicity and a number of authors have sought to construct mutants of those toxins that are free of toxic activity but that retain an adjuvant activity. However, there still exists a genuine need for effective mucosal adjuvants that are free of secondary effects.

In a first approach, the inventors sought to augment the adherence of liposomes to the mucosa, and thus to limit the quantities of antigen necessary for effective immunization, by coupling the antigen to liposomal FHA preparations. Adding FHA to liposomes containing an antigen such as Sm 28 GST from Schistosoma mansoni can augment the immune response against that antigen obtained nasally. The immediate explanation of that result was that, taking into account the adherence properties of the FHA, the latter allowed better adherence of liposomes to the mucosal surfaces and thus facilitated their access to cells of the immune system. That concept was behind International patent WO-A-98/16553, in which Mielcarek et al. describe hybrid constructions between all or a portion of FHA and all or a portion of an antigenic protein that was heterologous as regards FHA. The experimental results described therein led to the hypothesis that the immune response obtained as regards the heterologous protein resulted from the fact that FHA enabled better adhesion of liposomes to the mucosal surfaces. Those functional characteristics were used in the patent application supra to describe the use of FHA as a targeting molecule on the surface of different vaccine vectors (liposomes, microspheres, etc) to augment the immune response induced by the use of liposomes.

Hybrid proteins between FHA and glutathione S-transferase from Schistosoma mansoni (Sm 28 GST) resulted in an immune response as regards Sm 28 GST that protected against infection by Schistosoma mansoni (12).

The authors stated that adding FHA to liposomes that had already been produced with the antigen of interest alone produced satisfactory results; in other words, FHA, which contains hydrophobic sequences that enabled it to be readily inserted into liposomes and hydrophilic sequences, with FHA adhesin properties, enables both targeting of immunocompetent cells and of the different sites of the respiratory tract.

The authors deduced that it was possible and suggested the use of other synthetic vectors of the same type, for example vectors carrying a plurality of different antigens in addition to FHA to produce multi-antigenic vaccine formulations.

The foregoing indicates that FHA was used for its targeting properties.

Surprisingly, we have shown in the present invention that the FHA protein, or a polypeptide comprising a fragment of the FHA protein alone or included in a composition, is capable of inducing or augmenting an immune response against an immunogen or an antigen of interest when the FHA is present in a free form, i.e., not physically bonded to the antigen of interest against which the immune response is sought. This does not exclude a physical bond between any molecule or structure, either non antigenic, or for which a possible immune response is desired that is independent of the desired response, for example in the context of a vaccination.

The term “physically bonded” means that the FHA or its functional equivalent cannot be administered separately from the antigen of interest. Thus, this includes any type of direct or indirect bond with the antigen of interest. The term “direct bond” means that the antigen of interest and the FHA or FHA fragment are bonded covalently or non-covalently, but without the intervention of substances, vectors or particles. The tern “indirect bond”, in contrast, means any type of association and/or bond of FHA or an FHA fragment, of the antigen of interest with a molecule, a vector or any structure; non-limiting examples of said molecule, vector or structure include transporter molecules such as serum albumin, lipid structures such as liposomes, nano-particles, microspheres, ISCOMs etc. Indirect bonds can also be formed via covalent bonds, electrostatic bonds or hydrophobic type bonds.

One means for differentiating the notion of “physically bonded” from that of “free form” which characterizes the adjuvants of the invention is the capacity of the different elements of the association (antigen of interest, FHA or FHA fragment, vector or pharmaceutical vehicle, if appropriate) to be separated using the usual separation methods such as chromatography or electrophoresis.

It has been demonstrated that, in accordance with the invention, a composition containing an antigen of interest and FHA, in a form that is not physically bonded to the latter, is capable of inducing the production of seric antibodies and the production of specific antibodies of this antigen or immunogen in the mucosa. In the composition of the invention, the FHA is not physically bonded in the sense defined above to the other compounds or substances of the composition.

The compositions of the invention are immunogenic as regards the antigen contained therein and not as regards the FHA.

The Applicant has demonstrated that, surprisingly, the FHA protein possesses an activity adjuvanting the immune response when it is present in the free form, i.e., not physically bonded to the antigen of interest against which the immune response is desired.

The term “adjuvant compound” as used in the context of the present invention means a compound that can induce or enhance the specific immune response as regards an antigen or an immunogen, said immune response consisting equally of a humoral and/or cellular response. This immune response generally consists of stimulation of the synthesis of immunoglobulins specific to a given antigen, in particular IgG, IgA, IgM.

More surprisingly, it has been demonstrated that this adjuvant activity is manifested with efficacy during mucosal administration of the compositions.

Thus, in a first aspect, the invention consists of the use of the FHA protein as defined above in its free form for the preparation of a composition for adjuvanting the immune response.

Any protein such as FHA from B. bronchiseptica or that from B. parapertussis which has a similarity of at least 70% with the FHA of B. pertussis can also be used to prepare an adjuvant composition and forms a part of the invention.

Throughout the text, the term “similarity of X% with a reference sequence” means that X% of the amino acids are identical or modified by conservative substitution as defined in the ClustalW software for aligning amino acid sequences (http:///bioweb.pasteur.fr/docs/doc-gensoft/clustalw//) and that (100-X)% can be deleted, substituted with other amino acids, or (100-X)% can be added to the reference sequence.

In the present text, these proteins are considered to be functional equivalents of FHA as regards the adjuvant properties of the latter.

In a further aspect, the invention concerns an adjuvant composition for the immune response comprising the FHA protein or a functional equivalent thereof in the free form. Such an adjuvant composition can be administered to man or to an animal simultaneously with or sequential to the antigen of interest against which an immune response is sought. Advantageously, the adjuvant composition of the invention is administered simultaneously with the antigen of interest. It can also be administered a plurality of times, alone or in association with the antigen; in particular, it can be used in repeat treatment following immunization.

This adjuvant activity is manifested not only when the adjuvant and antigen are administered jointly in the same composition, but also when the antigen and adjuvant are administered separately, either using the same administration route, or using two different routes. As an example, the antigen can be administered systemically and the adjuvant mucosally or orally. Similarly, the number of administrations can differ for the antigen and adjuvant. Depending on the antigen and the selected immune response, the adjuvant can be administered once and the antigen a plurality of times, or vice versa

The invention also relates to immunogenic composition, characterized in that it comprises the adjuvant composition described above, in association with an immunogenic molecule or with an antigen, the immunogenic molecule or antigen not being physically bonded to the FHA protein or FHA protein fragment present in the adjuvant composition.

The term “antigen” as used in the present invention means any molecule or natural or synthetic structure whatever its nature (protein, saccharide or lipid, etc) recognized by the cells of the immune system and capable of activating them to induce a specific immune response against that antigen.

The term “immunogen” as used in the present invention means any composition that induces a strong immune response, particularly in the context of an immune protection against pathogenic organisms carrying said antigen.

It has been demonstrated that in accordance with the present invention, the FHA protein behaves as an adjuvant compound that can initiate or increase the immune response against different antigens or immunogens of various structures, recognized by the cells of the immune system in different manners.

The aim of adding an adjuvant to an antigen in a composition is to provoke or stimulate the immune response both in its primary phases (production of IgM) and in the secondary phases, namely the production of IgG in systemic cellular immunity, or IgA in mucosal immunity. This is particularly important in the case of the development of anti-infectious immunity. The adherence of microorganisms to the membranes of the epithelial cells of the mucosa is the first step in viral infection and bacterial colonization. IgA type antibodies can prevent adherence by coating the microorganism. They thus provide protection in external secretions such as tears, saliva or nasal secretions and in the intestinal and pulmonary mucus. Thus, it is easy to believe that if the FHA of the invention stimulates the IgA response, the impact on developing immunogenic compositions or vaccines must be enormous.

An immunogenic composition comprising the adjuvant of the invention and an immunogen or antigen is characterized in that the weight ratio of the adjuvant to the immunogen is in the range 10⁻⁴ to 10⁴, preferably in the range 0.03 to 300, more preferably in the range 0.4 to 5.

As indicated above, and demonstrated below, the adjuvant can be administered at different times from the antigen or using different administration routes, or associated in a single immunogenic composition.

In each case, the antigen can be of bacterial, viral or parasitic origin. It can also be an antigen that is specific to cancer cells, such as the embryo carcinoma antigen (ECA).

When the antigen is of bacterial origin, it can be a Bordetella, Shigella, Neisseria or Borrelia antigen, or diphtheria, tetanus or cholera toxins or toxoids.

When it is of parasitic origin, it can be a Plasmodium, Schistosoma or Toxoplasma antigen, for example.

In a further aspect, the invention provides a vaccine composition, characterized in that it comprises an adjuvant composition as described above, in association with a pharmaceutically acceptable vehicle.

The preparation of vaccine compositions containing a polypeptide as an immunogenic or antigenic molecule is well known to the skilled person and is in particular illustrated in U.S. patents U.S. Pat. No. 4,608,251, U.S. Pat. No. 4,601,903, U.S. Pat. No. 4,599,231, U.S. Pat. No. 599,230, U.S. Pat. No. 596,792 and U.S. Pat. No. 4,578,770, the contents of which are hereby incorporated by reference.

Such vaccine compositions are prepared in the form of injectable liquid solutions or suspensions or in the solid form, for example freeze-dried, adapted for dissolving prior to injection. The FHA or FHA portion used as the adjuvant and the antigen or immunogen are generally mixed with pharmaceutically acceptable excipients such as water, a saline buffer, dextrose, glycerol, ethanol, or mixtures thereof

A vaccine composition of the invention can also contain small quantities of auxiliary substances such as wetting agents or emulsifing agents, or buffers.

The vaccine compositions of the invention are formulated so that they are adapted to nasal, oral, subcutaneous, intradermal, intramuscular, vaginal, rectal, ocular or auricular administration.

The choice of auxiliary substances will be guided by the selected mode of administration. A preferred administration mode is nasal or oral administration.

The FHA and the antigen of interest can be formulated into a vaccine composition of the invention in a neutral or saline form.

Pharmaceutically acceptable salts include acid addition salts (formed with the free amino groups of the peptide) or those formed with inorganic acids such as hydrochloric acid or phosphoric acid or those formed with organic acids such as acetic acid, oxalic acid, tartaric acid or mandelic acid.

Salts formed with free carboxy groups can also be derived from inorganic bases such as sodium, potassium, ammonium, calcium or ferric hydroxides, or those formed with organic bases such as isopropylamine, trimethylamine, 2-ethylaminoethanol, histidine or procaine.

A vaccine composition of the invention includes a quantity of adjuvant and antigen that is effective on the immunogenic and/or therapeutic front. The respective quantities of adjuvant and antigen will depend on the individual to be treated, i.e., on the capacity of the immune system of the individual under consideration to develop an immune response, and on the required degree of protection.

Advantageously, the vaccine composition of the invention will comprise a dose of FHA or a functional equivalent in the range 0.1 to 1000 μg, advantageously in the range 10 to 300 μg, and highly preferably in the range 20 to 150 μg.

The quantity of immunogen or antigen included in a vaccine composition of the invention will depend both on the individual to be treated, on the type and on the nature of the antigen of interest.

By way of illustration, a vaccine composition of the invention will comprise 0.1 to 1000 μg of antigen or immunogen, preferably 1 to 300 μg and highly preferably 10 to 50 μg of antigen or immunogen.

Preferably, a vaccine composition of the invention will be administered once, and then repeated a few months after the initial administration.

The quantity of FHA or functional equivalent and antigen included in a vaccine composition of the invention can be adapted to obtain a good immune response, for example by monitoring in vitro proliferation of peripheral blood lymphocytes (PBL) cultivated in the presence of the antigen or immunogen, and more particularly by measuring the levels of cytokines secreted by the immune lymphocytes, or by determining the titers of seric and/or mucosal type antibodies produced.

Such tests can be carried out using conventional labels such as isotopic labels, or non-radioactive labels such as enzymes or fluorescent molecules.

Such techniques are well known to the skilled person and have in particular been described in U.S. Pat. No. 3,791,932, U.S. Pat. No. 174,384 and U.S. Pat. No. 3,949,064, said patents being hereby incorporated by reference.

The titers of seric and/or mucosal type antibodies can be measured as described in Examples 1 to 3 below.

In a particular embodiment of the vaccine compositions of the invention, such compositions will comprise, in addition to an effective quantity of a FHA protein or a functional equivalent, an immunologically effective quantity of at least two antigens or immunogens, for example three to twenty different antigens or immunogens, and preferably three to ten different antigens or immunogens.

Finally, the present invention pertains to a method for vaccinating or for stimulating cellular and humoral immunity consisting of using FHA or a functional equivalent thereof as defined above as an adjuvant in vaccination and/or for stimulating immunity. Such an adjuvant is administered in a composition in the free form, i.e., not physically bonded to the antigen or antigens against which an immune response is to be stimulated.

The antigen or antigens against which a response is desired are administered alone or in association with the adjuvant, without however being physically bonded to the latter. These compositions can also contain formulating elements that are adapted to the selected administration.

In this method of the invention, the adjuvant and antigen or antigens can also be administered simultaneously but in different compositions, or sequentially over time. In particular, the composition of the invention can be administered subsequent to an initial vaccination or an initial treatment, to reactivate the immune response against the antigen or antigens.

In a further aspect of the invention, the FHA or a functional equivalent thereof can be used as the active principle in preparing an immunostimulant composition.

The term “immunostimulant” means the property of stimulating total immunoglobulins, in particular total IgG or IgA. It is a non specific polyvalent response; this property is distinguished from that of the adjuvant for which only immunoglobulins specific to a given antigen are stimulated; this property is also distinguished from that of an immunogen such as a vaccine in which immunoglobulins specific to a given antigen are stimulated.

The invention pertains to an immunostimulant composition containing FHA or a functional equivalent thereof as the active principle and a pharmaceutically acceptable vehicle compatible with administration to man or to an animal.

The immunostimulant composition of the invention can advantageously be used to reinforce the immune defences of an organism in any pathological or prophylactic situation where this is required.

The pharmaceutical vehicle is selected as a function of the selected mode of administration. In addition to systemic routes, FHA or functional equivalents thereof as defined above is particularly suitable for oral or mucosal administration, and can thus be associated with different vehicles or excipients that are suitable for said administration routes.

Finally, the invention pertains to a drug for developing the immune defences of an organism and comprising FHA or a functional equivalent thereof as the active principle.

The results described in the experimental section below were obtained by nasal administration to mice with an adjuvant or with an immunostimulant in accordance with the invention. When an immunogenic composition comprising FHA in its non bonded form or a functional equivalent thereof is an antigen, the three antigen used under the experimental conditions described below were Sm 28 GST described above, and Megathura Crenulata haemocyanin (KLH).

The inventors have demonstrated that nasal administration of the antigen with FHA has the following properties:
a) under conditions in which the antigen alone is not capable of inducing a significant seric response, the presence of FHA induced a specific immune response in 7 out of 9 mice. Further, induction of an immune response directed against the antigen was not correlated with a response directed against FHA;
b) analysis of the antibodies present in bronchoalveolar lavage fluids (BALF) surprisingly indicated that whole FHA had an adjuvant effect on the production of antigen non-specific IgA in the bronchoalveolar liquid while there were no significant effects on the total quantity of IgG;
c) the results shown below indicate that this adjuvant activity for FHA is expressed against at least two different antigens. Further, this adjuvant activity persists using the systemic route: mice immunized subcutaneously with the antigen alone or the antigen and the adjuvant of the invention twice with a two-week interval responded better in the case when the adjuvant was present;
d) if previously vaccinated mice, for example vaccinated using DTCoq, were immunized nasally two months later with an adjuvant and antigen of the invention twice with a two week interval, it appears that the Dtcoq vaccination which caused the appearance of anti-FHA antibody did not prevent the induction of a response directed against the antigen.

The results shown in more detail below demonstrate that the adjuvant properties of free FHA are an intrinsic activity of said molecule, which is independent of a physical bond to the antigen as defined above; in other words, the adjuvant activity of said molecule is not linked to vectorization by the FHA of the antigen. In other words, FHA can constitute an adjuvant in immunogenic compositions or in vaccines and does not target the antigen towards immunocompetent cells.

Thus, FHA constitutes a novel effective adjuvant for mucosal administration representing an advantageous alternative to prior art adjuvants, whether they are adjuvants in the particulate form such as liposomes, microspheres, ISCOMS which are all synthetic globular structures encapsulating the administered antigen, or are recombinant microorganisms expressing an antigen of interest.

Further, FHA is characterized by a total innocuousness and does not cause the secretion of pro-inflammatory cytokines on a local level, in contrast to the most effective currently known mucous adjuvants which are bacterial toxins, such as cholera toxin, E. coli toxin or Bordetella pertussis toxin.

Identifying FHA or a functional equivalent thereof in accordance with the invention as an immune response adjuvant, in particular the mucosal immune response, thus fulfils a genuine need in the prior art, for an effective mucous adjuvant that is free of toxicity.

Further, the adjuvant activity of the FHA is not restricted by or dependent on a given haplotype of the histocompatibility antigen. In contrast, the adjuvant properties of these proteins is observed whatever the MHC haplotype, as demonstrated in the experiments carried out on genetically heterogeneous mice such as “Outbred” mice, for example OF1 mice.

An analysis of the different isotypes of the seric antibody produced as a response to immunization against these antigens in the presence of FHA has shown that the quantitative ratio between the different IgG isotypes was not significantly different when the antigen was administered simultaneously with the FHA.

Further, an analysis of seric antibody isotypes shows a predominance of IgG1 and IgG2a isotypes in response to administration of Sm28GST or KLH in the presence of FHA.

These results indicate that the isotype profile of the specific antibodies produced essentially depends on the antigen used, FHA not in itself inducing polarization.

The results obtained and described in detail in the experimental section show that FHA can be used as an adjuvant as such, whatever the histocompatibility haplotype of the individual to be immunized, and whatever the nature of the antigen or immunogen against which a mucosal and/or systemic type immune response is sought.

Such a polypeptide can be obtained by genetic recombination as disclosed by Brown et al. (2), Reiman et al., (3) or Delisse-Gathoye (4).

In a particular embodiment of the FHA protein or a functional equivalent thereof in accordance with the invention, a polynucleotide coding for FHA or its functional equivalent is inserted into an expression vector comprising at least one promoter and a terminator required for expression of the polynucleotide in an appropriate host cell.

The polynucleotide coding for a functional equivalent of the FHA as defined above has a similarity of at least 70% with the sequence coding for the FHA and described in (2).

The term “similarity” means that, for the same reading frame, a given triplet is translated by the same amino acid. This term thus includes modifications to the bases resulting from degeneracy of the genetic code.

The similarity percentage is determined by comparing a given sequence with the reference sequence. When they are of different lengths, the similarity percentage is based on the percentage of nucleotides in the shorter sequence that are similar to those of the longer sequence.

The degree of similarity can be conventionally determined using programs such as ClustalW (Thompson et al., Nucleic Acids Research 22 (1994), 4673-4680) distributed by Julie Thompson (Thompson@EMBL-Heidelberg.de) and Toby Gibson (Gibson@EMBL-Heidelberg.de) at the European Molecular Biology Laboratory, Meyerhofstrasse 1, D-69117 Heidelberg, Germany. ClustalW can also be downloaded from a number of websites including IGBMC (Genetics and Molecular and Cellular Biology Institute, B P 163, 67404 Illkirch Cedex France; ftp://Rp-igbmc.u-strabg.fr/pub/) and EBI (ftp://ftp.ebi.ac.uk/pub/software/) and any sites for the Bioinformatics Institute, Wellcome Trust Genome Campus, Hinxton, Cambridge CB10 ISD, UK.

An expression vector comprising such polynucleotides will also advantageously comprise at least one functional origin of replication in the host cell in which expression of the FHA protein or a portion of the recombinant FHA is sought, and at least one selection marker such as an antibiotic resistance marker, for example neomycin, tetracycline, rifampicin or ampicillin.

An appropriate host cell can equally be of either bacterial or eukaryotic origin.

To construct such expression vectors and transform or transfect appropriate host cells, the skilled person can advantageously refer to Sambrook's work (2001): “Molecular Cloning: A Laboratory Manual”, 3^{rd} edition, Cold Spring Harbor Laboratory, Cold Spring Harbor, N.Y.

The recombinant FHA protein or its functional equivalent can be purified using techniques that are well known to the skilled person.

The FHA protein can also be prepared using conventional chemical synthesis techniques, equally in a homogeneous solution or in the solid phase.

By way of illustration, the skilled person can find such techniques for chemically synthesizing polypeptides in Houbenweyl (1974), “Methode der Organischen Chemie”, E. Wunsh Ed., Vol. 15-I and 15-II, Thieme, Stuttgart, or the Merrifield technique (1965a; 1965b); Merrifield R B (1965a), Nature, 207 (996): 522-523, Merrifield (1965b), Science, 150 (693): 178-185.

The FHA protein described above can also be used to prepare a composition adjuvanting an immune response when certain amino acids are substituted by conservative substitution. The term “conservative substitution” means substitution of an amino acid by another with no consequence or minor consequences on the tertiary structure of the sequence and on the hydrophobic nature of that sequence. By way of example, substitution of a guanine by an alanine or vice-versa is qualified as conservative substitution. In the same manner, valine, leucine, isoleucine are amino acids that can be substituted mutually in a conservative manner. Non-limiting examples of other conservative substitution groups are (D,E), (K,R), (N,Q), and (F,W,Y).

In a particular embodiment of a polypeptide containing all or a portion of the mature FHA protein, said polypeptide can advantageously be rendered resistant to proteolysis, for example by replacing the specific peptide bond —CONH— by a reduced bond —CH₂NH—, a retro-inverted bond —NHCO—, a methylene-xy bond —CH₂O—, a thiomethylene bond —SH₂—S—, a carba bond —CH₂CH₂—, a ketomethylene bond —O—CH₂—, a hydroxyethylene bond —CHOH—CH₂—, a —N—N— bond, an E-alkene bond or a —CH═CH— bond.

In a further embodiment, in the polypeptide comprising all or a portion of he amino acid sequence for the FHA protein, one or more amine acid resides of the L form can be replaced by the corresponding amino acid in the D form; a glutamic acid residue could be replaced by a pyro-glutamic acid residue. The synthesis of peptides containing at least one amino acid residue in the D form is described, for example, by Koch Y, (14).

In a further aspect, the immunogenic composition of the invention is characterized in that the antigen of interest is of human, vegetable or animal bacterial, viral or parasitic origin.

The present invention will now be illustrated by the following non-limiting figures and examples:

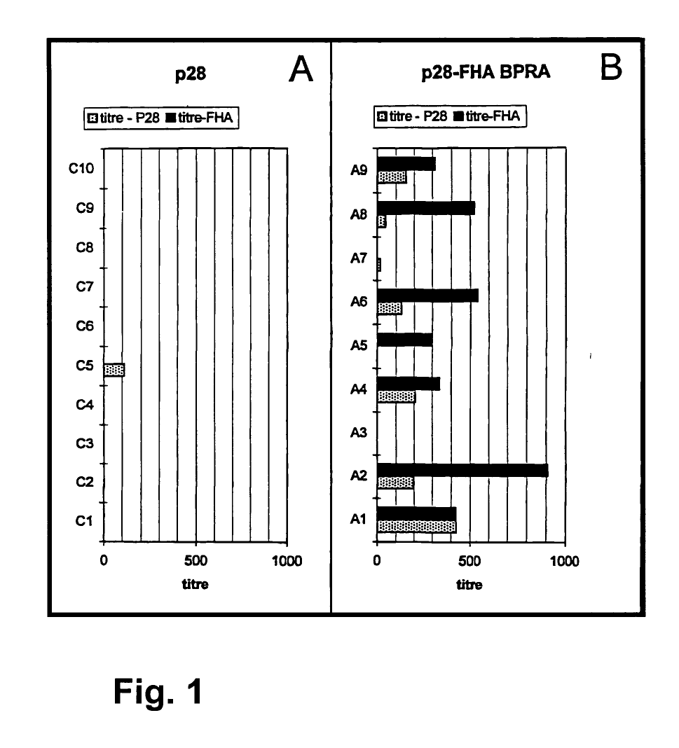

FIG. 1 shows the titers of seric antibodies specific to the Sm28GST or FHA protein, after immunizing mice against the Sm28GST protein in the presence or absence of FHA.

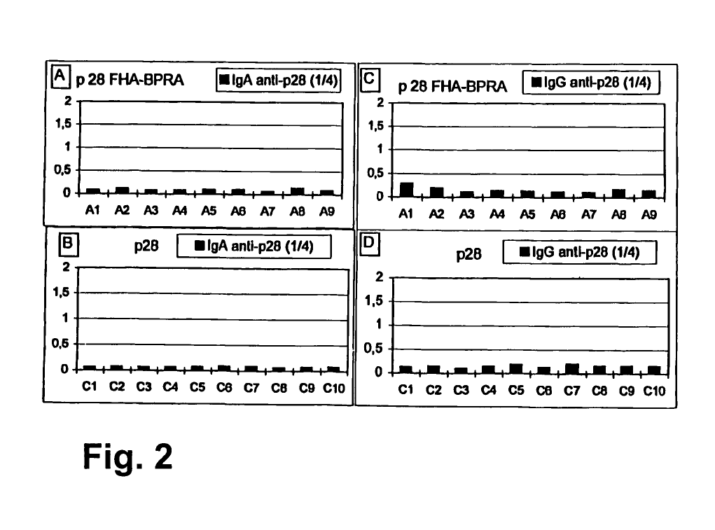

FIG. 2 shows the optical densities observed for a ¼ dilution of IgA and IgG isotype antibodies in bronchoalveolar lavage fluids from mice immunized against Sm28GST in the presence or absence of FHA.

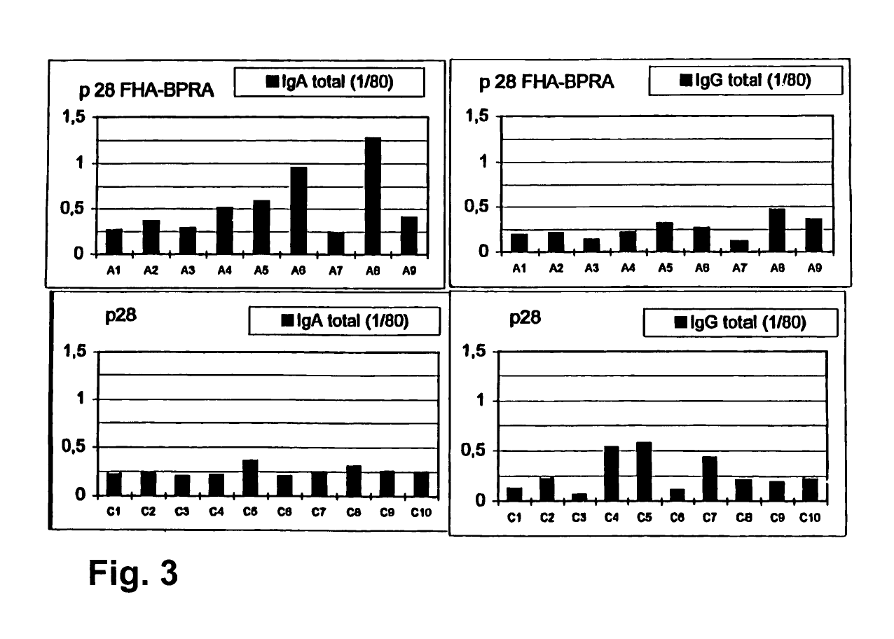

FIG. 3 shows the optical densities observed for a 1/80 dilution of total IgA and IgG isotype antibody in bronchoalveolar lavage fluids from mice immunized with the protein Sm28GST in the presence or absence of FHA.

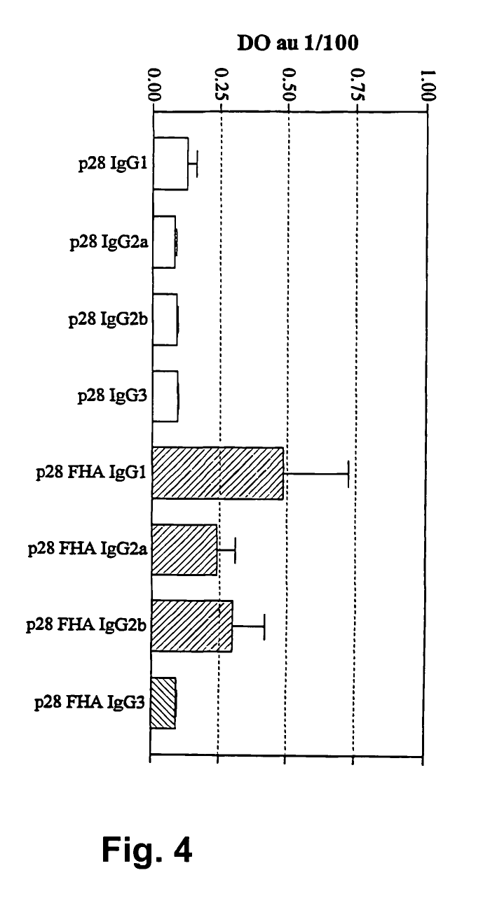

FIG. 4 shows the isotype profile of seric antibodies specific to Sm28GST in mice immunized against this protein in the presence and absence of FHA.

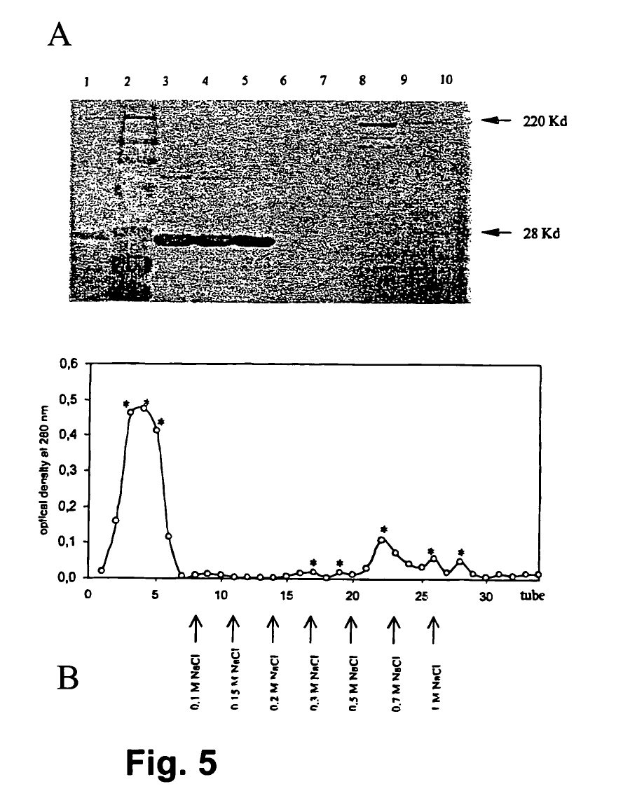

FIG. 5 shows a selective elution of FHA and the Sm28GST protein from a mixture of the adjuvant and that antigen.

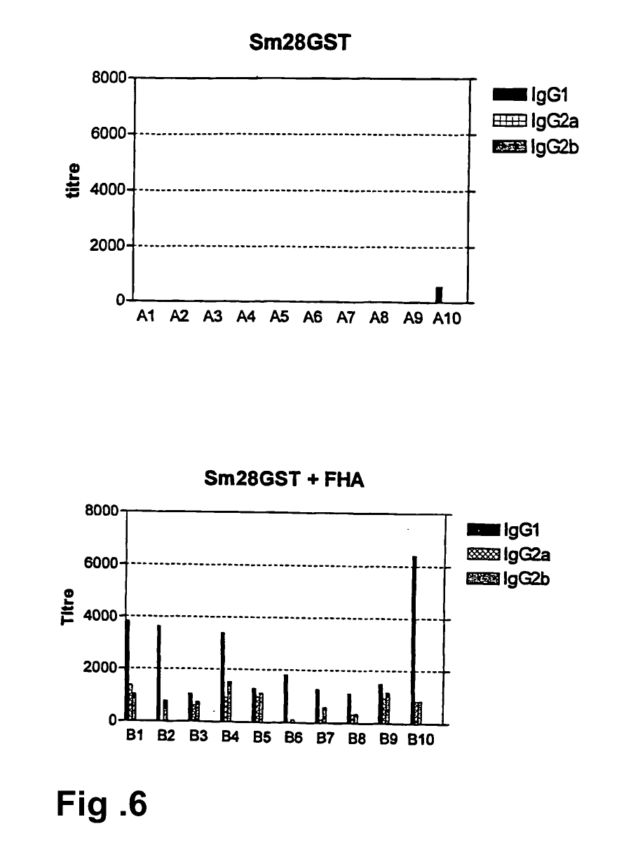

FIG. 6 shows the specific anti Sm28GST response after vaccination with Dtcoq.

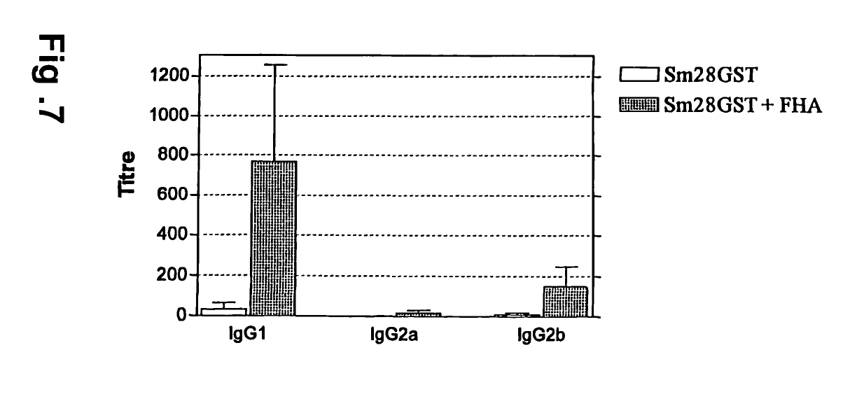

FIG. 7 shows the antibody titers obtained after subcutaneous administration of the Sm28GST antigen with or without FHA after immunizing twice with a two week interval. The seric response was observed one week later.

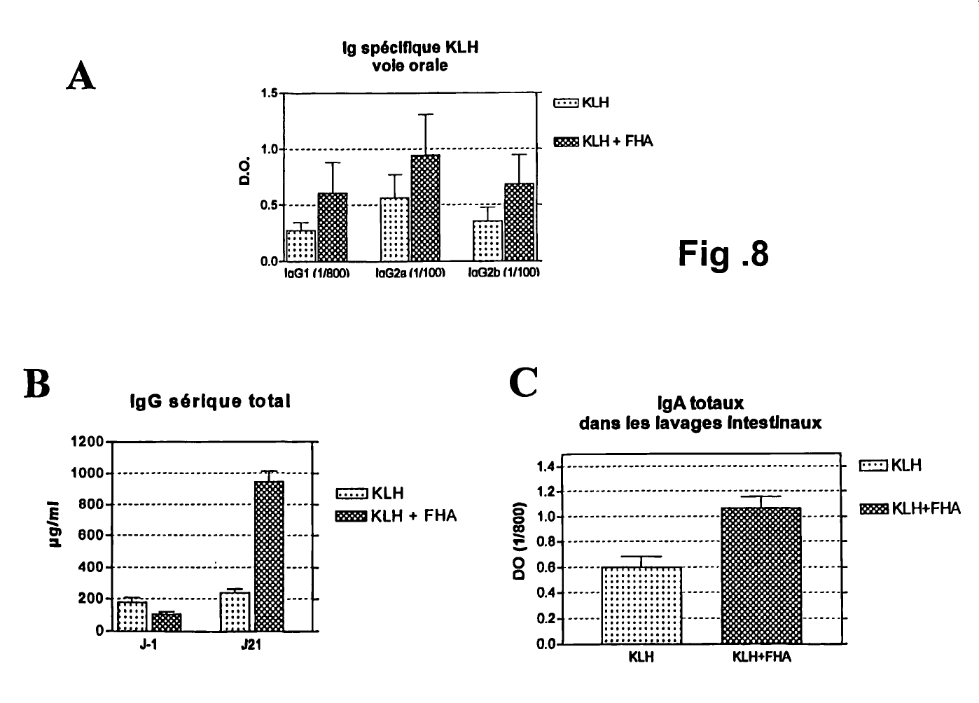

FIG. 8 shows the adjuvant and immunostimulant effect of FHA after oral administration to three groups of mice that have received at two week intervals:
30 μg of KLH;
30 μg of KLH+5 μg of whole FHA.

FIG. 8a shows the observed optical density at D21 for specific IgG1, IgG2a and IgG2b of KLH in serums for respective dilutions of 1/800, 1/100 and 1/100.

FIG. 8b shows the observed optical density at D21 for non specific total IgG in serums for a dilution of 1/10000.

FIG. 8c shows the observed optical density at D21 for non specific total IgA in intestinal lavage fluid a dilution of 1/800.

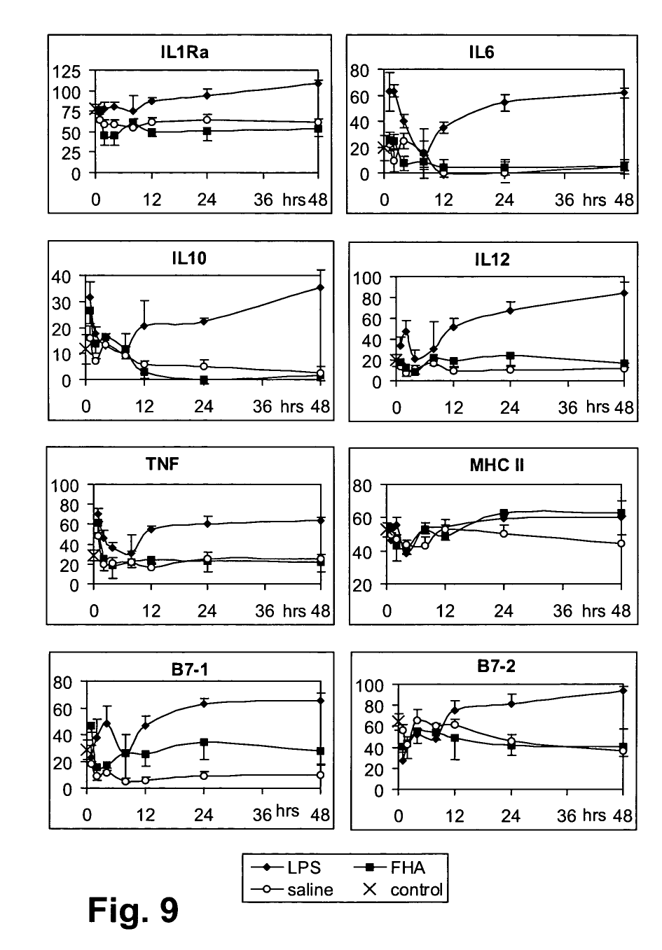

FIG. 9 shows the analysis of mRNA analysis locally induced in the lung after administration of either apyrogenic saline solution or 5 μg of FHA, i.e., 20 μg of LPS.

### EXAMPLES

### A. Materials and Methods

Antigens

Glutathion S-transferase from Schistosoma mansoni (Sm28GST) was supplied by Société Transgène (Strasbourg, France). FHA was purified by heparin-Sepharose column chromatography as described previously (Menozzi et al., FEMS 1991) from Bordetella pertussis RA [BPRA] supernatants, a strain that is free of the pertussis toxin gene.

The FHA preparation was eluted on an Acticlean [Sterogène] column to eliminate endotoxin contaminants. The final endotoxic activity was then evaluated using a Limulus test (Biowhittaker). Megathura crenulata haemocyanin (KLH) was purchased from Calbiochem.

Immunizations

6 week old OF1 mice (Iffa Credo, L'Arbresie, France) were anaesthetized intraperitoneally with 200 μl of sodium pentobarbital (5%; Sanofi, Liboume, France) per 10 g of body weight. The mice were immunized nasally with 40 μl of apyrogenic saline solution into which the different antigenic preparations had been dissolved. The mice were then instilled twice with a two week interval and sacrificed one week later to take the blood samples and make the bronchoalveolar washes. The doses of antigen employed are described in the examples.
Sample Recovery and Analysis

The mice were bled at the tail (or by cardiac puncture on the day of sacrifice) and the serum was preserved at −20° C. until the day of analysis. The bronchoalveolar lavage fluid (BALF) was collected by cannulizing the trachea and resulted from lavage of the lungs with 0.5 ml of PBS. After centrifuging for 10 min at 4000 g to eliminate cells and tissue debris, the BALF was then frozen at −20° C. after adding phenylmethylsulphonyl fluoride at a final concentration in 1 mM.

The amount of antibody in the serum and in the BALF was determined using ELISA. Microplates (Maxisorp, Nunc) were incubated overnight at 4° C. with 50 μl/well of a solution of FHA (5 μg/ml) or Sm28GST (10 μg/ml), or KLH (5 μg/ml). The serum, diluted in PBS containing 1‰ of Tween-20 and 5‰ of gelatine (PBS/Tw/g) was then added to the plates after rinsing 4 times with PBS containing 1‰ of Tween-20 (PBS/Tw). After incubating overnight at 4° C., the plates were rinsed 4 times with PBS/Tw then incubated with different dilutions of antibody (in PBS/Tw/g; 1 h30 at 37° C.) conjugated with peroxisase (anti-mouse IgG (H+L) or IgG1, or IgG2a or IgG2b or IgG3; Southern Biotechnologies Associates, Birmingham, USA). After rinsing 4 times with PBS/Tw, the plates were revealed with a 1 mg/ml solution of ABTS (Sigma) in citrate buffer (0.1 M, pH 4) and containing 0.03% of H₂O₂. The optical density was measured at 405 nm (Titertek Multiscan MCC/340) after 30 minutes. Isotype IgA antibodies were detected after incubating with a biotinned mouse anti-IgA antibody (Zymed) diluted in PBS/Tw/g for 1 h30 at 37° C. After rinsing 4 times with PBS/Tw, the plates were incubated for 30 minutes at 37° C. in the presence of streptavidin coupled to peroxidase (Amersham) diluted in PBS/Tw/g. After rinsing 6 times with PBS/Tw, the plates were revealed with an OPD solution (1 mg/ml; Sigma) for 30 minutes at 37° C. The reaction was stopped with a 2N HCl solution and the optical density was measured at 492 nm. The regression line giving the logarithm of the observed optical density as a function of the inverse of the dilution was calculated. By extrapolating that line, the titers corresponded to the inverse of the dilution for which the optical density was three times the value of the optical density obtained with PBS.

Antibodies were detected in the BALF in the same manner with a few modifications. After adsorption of the antigen, the plates were saturated with a gelatin solution (5% PBS) for 30 minutes at ambient temperature. The BALF and the antibodies were then diluted with PBS/Tw. The concentrations of total IgA and total IgG in the BALF were then evaluated for microplates into which non-labelled anti-mouse IgA or IgG antibodies had been adsorbed and by comparison with a reference curve produced using purified mouse myelomatous IgA or IgG (Sigma).
Binding Test

Sepharose beads coupled to heparin (CL-6B; Pharmacia) were re-suspended in PBS and packed into a column (1 cm diameter, 1.3 ml of gel). After rinsing the column, a solution containing Sm28GST (200 μg) and FHA (40 μg) was deposited.

After rinsing, increasing concentrations of NaCl were deposited on that column (0.1 M NaCl up to 1 M). Analysis of the samples recovered after passage over the column then eluting was carried out by acrylamide gel electrophoresis after precipitating with TCA.
Determination of Messenger mRNA

The mice were immunized as before, nasally (volume=50 μl) either with apyrogenic physiological serum or with 5 μg of FHA, or with 20 μg of LPS (Sigma). Non-dosed mice were used as the control. The mice were sacrificed at different times (between 1 h and 48 h), the whole lungs were removed and ground in a solution of RNAzol® solution. The RNA was extracted using chloroform then precipitated with isopropanol. After washing, the RNA residue was taken up in suspension in water. Reverse transcription was carried out to synthesise the cDNa corresponding to the extracted RNA. Polymerase chain reaction experiments were carried out on all of the extracts to amplify the specific DNA fragments of different markers with the pairs of primers indicated in the table below:

| | |
|---|---|
| CYTOKINE | SEQUENCES |

| | |
|---|---|
| IL1 Ra | sense: |
| | 5′ AGA CCC TGC AAG ATG CAA GCC TTC AGG 3′ |
| | antisense: |
| | 5′ GGT CAG CCT CTA GTG TTG TGC AGA 3′ |
| IL6 | sense: |
| | 5′ GTG ACA ACC ACG GCC TTC CCT ACT 3′ |
| | antisense: |
| | 5′ ; GGT AGC TAT GGT ACT CCA 3′ |
| IL10 | sense |
| | 5′ CGG GAA GAC AAT AAC TG 3′ |
| | antisense: |
| | 5′ CAT TTC CGA TAA GGC TTG G |
| IL12 | sense |
| | 5′ GAC CCT GCC CAT TGA ACT GGC 3′ |
| | antisense |
| | 5′ CAA CGT TGC ATC CTA GGA TCG 3′ |
| TNFα | sense |
| | 5′ AGC CCA CGT CGT AGC AAA CCA CCA A 3′ |
| | antisense |
| | 5′ ACA CCC ATT CCC TTC ACA GAG CAA T 3′ |
| MHC II | sense |
| | 5′ TGT CCA GGA CAG AGG CCC TC 3′; |
| | antisense |
| | 5′ TCC ACA TGG CAG GTG TAG AC 3′ |
| B7-1 | sense |
| | 5′ GTA TTG CTG CCT TGC CGT TA 3′ |
| | antisense |
| | 5′ ATG GTG TGG TTG CGA GTC GT 3′ |
| B7-2 | sense |
| | 5′ AGG ACA TGG GCT CGT ATG AT 3′ |
| | antisense |
| | 5′ GAA CAC ACA CAA CGG TCA TA 3′ |

The amplification products were visualized using ethidium bromide after migration on agarose gel. The bands obtained were analyzed by an image analysis technique and an index corresponding to the intensity of those bands was drawn up.

### EXAMPLE 1

Study of Adjuvant Activity of FHA as Regards an Immune Response Against the Sm28GST Protein From Schistosoma mansoni

OF1 (outbred) mice received a nasal administration of 50 μg of Sm28GST in the presence or absence of 5 μg of FHA diluted in the same sample, twice with a two week interval.

The production of seric IgG isotype antibody specific for Sm28GST or FHA one week after the second nasal instillation was analysed and the results are shown in FIG. 1.

The titer of seric IgG antibody directed against Sm28 (grey bars) and the FHA protein (black bars) was measured in serum from mice that had respectively received the Sm28GST protein (FIG. 1A), and a Sm28GST+FHA mixture (FIG. 1B).

These results indicate that the Sm28GST protein alone was not capable of inducing a significant seric antibody response. In contrast, the presence of FHA allowed induction of a specific immune response against Sm28GST in seven out of nine mice. Further, an analysis of the response directed against FHA in these animals showed that induction of an immune response directed against Sm28GST was not necessarily correlated with a specific response directed against FHA.

### EXAMPLE 2

Study of Adjuvant Activity of FHA on the Production of Antibodies by the Immune Cells of the Respiratory Tract

OF1 mice were immunized in accordance with the protocol described in Example 1 and the production of total or specific isotype IgG and IgA antibodies for the Sm28GST protein contained in the bronchoalveolar lavage liquids was analyzed. The results are shown in FIGS. 2 and 3.

The titers of specific antibodies for the protein Sm28GST (FIG. 2) or total antibody (FIG. 3) in the bronchoalveolar ravage liquid for isotype IgA (A, B) or IgG (C, D) were measured in mice 21 days after the second nasal instillation of Sm28GST (B, D) and Sm28GST+FHA (A, C).

The results shown in FIG. 2 indicate that the presence of FHA during immunization had not induced a detectable production of specific Sm28GST antibodies in the bronchoalveolar lavage liquid.

The results of FIG. 3 show the substantial effect of FHA on the quantity of total isotype IgA antibody contained in the bronchoalveolar lavage liquid, while there does not appear to have been a significant effect on the total quantity of isotype IgG antibody.

### EXAMPLE 3

Study of Polarization of Adjuvant Activity of FHA: Istoype Response

The immunization protocols were identical to those described for Examples 1 and 2; nasal instillations were carried out in the mice using a mixture containing 50 μg of antigen and 5 μg of FHA respectively.

The titers of specific antibodies for the antigen or immunogen of interest were measured 21 days after the second nasal instillation into mice that had received that antigen or immunogen respectively. The quality of the different isotypes IgG1, IgG2a, IgG2b and IgG3 was measured.

The antigen used was the Sm28GST protein from Schistosoma mansoni.

The results shown in FIG. 4 show, as demonstrated in Examples 1 and 2, the adjuvant activity of whole FHA. An analysis of the profile of the different IgG antibody isotypes shows a quantitatively similar production of antibodies with isotypes IgG1 and IgG2a that are specific to the Sm28GST protein, significant of a “mixed” immune response.

### EXAMPLE 4

FHA and the Antigen Are Not Physically Bonded In An Immunogenic Composition of the Invention

In order to determine the existence of a non covalent physical bond between the antigen of interest and the FHA, of a nature so as to constitute a complex, a mixture of FHA and Sm28GST was prepared as indicated in the “Materials and Methods” section.

The mixture of FHA and Sm28GST was deposited on a heparin column. Elution was carried out with increasing concentrations of NaCl, each of the elution fractions then being analyzed for its protein concentration (FIG 5b).

Fractions also underwent electrophoretic migration in a polyacrylamide gel in the presence of SDS simultaneously with a series of protein molecular weight markers (FIG. 5a).

The results show that elution in an increasing gradient of NaCl desorbed the FHA from a concentration of 0.5 M of NaCl (see FIG. 5b). Further, an analysis of the nature of the proteins present in the different elution fractions showed that no trace of the Sm28GST protein could be detected (FIG. 5a).

As a result, this shows a total absence of physical covalent or non covalent interaction between the FHA and the antigen of interest.

### EXAMPLE 5

Adjuvant Activity of FHA Persists in Vaccinated Subjects

OF1 mice were vaccinated by subcutaneous injection of 50 μl of the Dtcoq gene (commercial vaccine). Two months later, they were immunized nasally, twice with a two week interval. An analysis of the seric response obtained one week later showed that vaccination with Dtcoq, which caused the appearance of circulating anti-FHA antibody, had not prevented the induction of a response directed against Sm28GSt in the group co-administered with FHA (FIG. 6).

The experiments of Examples 5 and 6 confirmed the intrinsic adjuvant power of FHA by a systemic route and its potential use in vaccinated populations.

### EXAMPLE 6

The Adjuvant Activity of FHA Persists Using the Systemic Route

OF1 mice were immunized subcutaneously, i.e., with 50 μg of smGST, twice with a two week interval. An analysis of the seric response obtained one week later showed the induction of a response directed against Sm28GST in the two groups that had been co-administered with FHA (FIG. 7). This response, however, remained weaker than that obtained nasally.

### EXAMPLE 7

The adjuvant activity of FHA persists using the oral route

OF1 mice were immunized orally either with 30 μg of KLH or with 30 μg of KLH and 5 μg of FHA, twice with a two week interval. An analysis of the seric response obtained one week later showed the induction of a response directed against KLH in the group co-administered with FHA (FIG. 8A).

For oral administration, the protein or proteins were dissolved to the required concentration in a solution of PBS containing 30 g/l of NaHCO₃. A volume of 200 μl was administered to non anaesthetized animals using a gastric probe.

Intestinal lavages were carried out at D21 after cervical rupture of the animals using a modification of the technique described by Nedrud et al. (1987). The intestine was sectioned below the stomach and above the caecum and rinsed with BPBS. It was then slit along its length. The intestine and its contents were re-suspended in 2 ml of buffer followed by 25 mM of NaCl, 40 mM of Na₂SO₄, 10 mM of KCl, 20 mM of NaHCO₃, 50 mM of EDTA, 162 mg/ml of polyethylene glycol (MW 3350) and 1‰ of aprotinin. After centrifuging, the supernatants were frozen after adding 1 mM PMSF.

From this point on, the material and methods used in particular when assaying antibody were identical to those used above.

### EXAMPLE 8

FHA Can Cause a Polyclonal Activation of Plasmocytes Which Generates Antibody Production

8.1. Nasal administration:

FIG. 3 of Example 2 illustrates the amount of non specific total antibody detected in the bronchoalveolar lavage liquid after nasal administration of Sm28GST in the presence or absence of FHA or FHA44.

As indicated above, the results of FIG. 3 show that FHA44 induces massive quantities of total isotype IgA and IgG antibody in the bronchoalveolar secretions which cannot be uniquely correlated with the appearance of specific antibodies.

8.2. Oral administration:

An analysis of the total immunoglobulins of the samples obtained during the oral administration experiment was carried out. A substantial increase in the total seric IgG was observed in the group that had received FHA (FIG. 8B). Further, FHA was capable of increasing the amount of non specific IgA in the intestinal lavage liquid (FIG. 8B) even though no specific response could be detected.

This non specific activity of FHA and its derivatives indicates that these bacterial products are capable of stimulating general immunity in the organism.

It appears that while FHA is particularly effective as an adjuvant for a specific response (FIG. 8a), FHA appears to be a better immunostimulant for non specific IgG.

### EXAMPLE 9

FHA locally induces an increase in MRNA coding for the major histocompatibility complex MHCII and for the co-stimulation molecule B7-1

OF1 mice were administered nasally with 5 μg of FHA or 20 μg of LPS or with apyrogenic physiological water alone. After different periods (1 h, 2 h, 4 h, 8 h, 12 h, 24 h, 48 h), the mice were sacrificed and the lungs were removed. RT-PCR analysis of the MRNA of these extracts demonstrated an increase in the mRNA coding for the major histocompatibility complex MHCII and for the co-stimulation molecule B7-1 in the FHA group compared with the physiological water group. In contrast, the level of mRNA expression for the different cytokines studied showed no difference between these two groups while a substantial increase was observed in the LPS control group.

The results are shown in FIG. 9.

The increase in MHCII and B7-1 suggests an increase in local presentation induced by FHA which could be partially explained by its adjuvant activity. Further, it can be seen that the absence of overexpression of pro-inflammatory cytokines induced by FHA is a supplemental quality of this molecule when used as an adjuvant.

### REFERENCES

(1) LOCHT, C., BERTIN P., MENOZZI F. D., and RENAUD G. (1993).

The filamentous haemagglutinin, a multifaced adhesin produced by virulent . Bordetella sp. Mol. Microbiol. 9: 653-660.

(2) BROWN. D. R., and PARKER. C. D. (1987). Cloning of the filamentous hemagglutinin of Bordetella pertussis and its expression in Escherichia coli. Infect. Immun., 55:154161.

(3) RELMAN D., DOMENIGHINI, M., TUOMANEN, E., RAPPUOLI, R. and FALKOW, S. (1989).

Filamentous hemagglutinin of Bordetella pertussis: nucleotide sequence and crucial role in adherence. Proc. Natl. Acad. Sci. USA. 86:2637-2641.

(4). DELISSE-GATHOYE, A. M., LOCHT C., JACOB, F., RAASCHOU-NIELSEN, M., HERON, I., RUELLE. J, L., DE WILDE, M., and CAB EZON, T. (1990). Cloning, partial sequence, expression and antigenic analysis of the filamentous hemagglutinin gene of Bordetella pertussis. Infect. Immun. 58:2895-2905.

(5) MAKHOV, A. M., HANNAH, J. H., BRENNAN, M. J., TRUS, B. L., KOCSIS, E., CONWAY, J. F., WINGFIELD, P. T., SIMON, M. N., and STEVEN, A. C. (1994). Filamentous hemagglutinin of Bordetella pertussis; a bacterial adhesin formed as a 50 nm monomeric rigid rod based on a 19-residue repeat motif rich in β-strands and turns. J. Mol. Biol, 241:110-124.

(6) RENAULD-MONGENIE, G., CORNETTE, J., MIELCAREK, N., MENOZZI, F. D. and LOCHT, C. (1996). Distinct roles of the N-terminal and C-terminal precursor domains in the biogenesis of the Bordetella pertussis filamentous hemagglutinin. J. Bacteriol. 178:1053-1060.

(7) GREGORIADIS G. (1990). Immunological adjuvants: a role for liposomes. Immunol. Today 11: 89-97.

(8) ELDRIDGE, J. H., GILLEY, R. M., STAAS, J. K., MOLDOVEANU, Z., MEULBROEK, J. A., and TICE, T. R. (1989). Biodegradable microspheres: vaccine delivery system for oral immunization. Curr. Top. Microbiol. Immunol. 146: 59-66.

(9) MOWAT, A. M. and DONACHIE, A. M. (1991). ISCOMs-a novel strategy for mucosal immunization? Immunol. Tod. 12: 383-385.

(10) RENAULD-MONGENIE, G.. MIELCAREK, N., CORNETTE J., SCHACHT, A. M., CAPRON, A., RIVEAU, G., and LOCHT C. (1996). Induction of mucosal immune responses against a heterologous antigen fused to filamentous hemagglutinin after intranasal immunization with recombinant Bordetella pertussis. Proc. Natl. Acad. Sci. USA 93; 7944-7949.

( 11) MIELCAREK, N., RIVEAU; G., REMOUE, F., ANTOINE, R., CAPRON, A., and LOCHT, C. (1998). Homologous and heterologous protection after single intranasal administration of live attenuated recombinant Bordetella pertussis. Nature Biotech. 16: 454-457.

(12) POULAIN-GODEFROY, O., MIELCAREK, N., IVANOFF, N., REMOUE, F., SCHACHT, A. M.; PHILLIPS, N., LOCHT, C., CAPRON, A. and RIVEAU, G. (1998). Enhancement of immunogenicity by intranasal antigen delivery using liposomes bearing the Bordetella pertussis filamentous hemagglutinin. Infect. Immun. 66: 17641767.

(13) HANNA. J. H., MENNOZI, F .D., RENAULD, G., LOCHT, C. and BRENNAN, M. J. (1994). Sulfated glycoconjugate receptors for the Bordetella pertussis adhesin filamentous hemagglutinin (FHA) and mapping of the heparin-binding domain on FHA. Infect. Immun, 20 62: 5010-5019.

(14) KOCH Y., BARAM, T., HAZUM, E.; and FRIDKIN, M. (1977). Resistance to enzymic degradation of LH-RH analogues possessing increased biological activity. Biochem. Biophys. Res. Commun., 74:488-491.

1. Use of the FHA protein or a functional equivalent thereof in its free form for the preparation of a composition adjuvanting the immune response.

2. Use of the FHA protein or a functional equivalent thereof in preparing an immunostimulant composition.

3. Use of a sequence according to claim 1 or claim 2 obtained by conservative substitution of amino acids.

4. Use of a sequence according to claim 1 or claim 2 rendered resistant to proteolysis by replacing the peptide bond —CONH— by a reduced bond —CH₂NH—, a retroinverso bond —NHCO—, a methyleneoxy bond —CH₂—, a thiomethylene bond —SH₂—S—, a carba bond —CH₂CH₂—, a ketomethylene bond O—CH₂—, a hydroxyethylene bond —CHOH—CH₂—, a —N—N bond, an E-alkene bond or a —CH═CH— bond.

5. Use according to any one of the preceding claims in which the adjuvant is an amino acid sequence having a homology of at least 70% with FHA.

6. A composition adjuvanting the immune response comprising the FHA protein in its free form, or a derived sequence in accordance with one of claims 3 to 5.

7. An immunogenic composition, characterized in that it comprises an adjuvant according to claim 6, in association with an immunogenic molecule or with an antigen.

8. An immunogenic composition according to claim 7, characterized in that the weight ratio of the adjuvant and the immunogen is in the range 10⁻⁴ to 10⁴, preferably in the range 0.03 to 300, and more preferably in the range 0.4 to 5.

9. An immunogenic composition according to claim 7, characterized in that the antigen is of bacterial, viral or parasitic origin.

10. An immunogenic composition according to claim 7, characterized in that the antigen is selected from Bordetella, Shigella, Neisseria, Borrelia antigens, or from diphtheria, tetanus or cholera toxins or toxoids.

11. An immunogenic composition according to claim 7, characterized in that the antigen is a viral antigen.

12. An immunogenic composition according to claim 7, characterized in that the antigen is a parasitic antigen, in particular of Plasmodium, Schistosoma or Toxoplasma.

13. A vaccine comprising an immunogenic composition according to one of claims 7 to 12, in association with a pharmaceutically acceptable vehicle.

14. A vaccine according to claim 13, characterized in that the vehicle is compatible with nasal administration.

15. A vaccine according to claim 13, characterized in that the vehicle is compatible with oral administration, subcutaneous administration or intravenous, intradermal or intramuscular administration.

16. A vaccine according to claim 13, characterized in that the vehicle is compatible with rectal, vaginal, ocular or auricular administration.

17. An immunostimulant composition containing FHA or a functional equivalent thereof as the active principle and a pharmaceutically acceptable vehicle compatible with administration to man or to animals.

18. A composition according to claim 17, for oral administration.

19. A composition according to claim 17, for mucosal administration.
